(19) United States
(12) Patent Application Publication
(10) Pub. No.: US 2011/0015751 A1
(43) Pub. Date: Jan. 20, 2011

(54) Augment For Use In A Knee Prelacement System And Method Of Performing A Knee Replacement Operation

(76) Inventor: Lindsay Laird, New South Wales (AU)

Correspondence Address: INSKEEP INTELLECTUAL PROPERTY GROUP, INC, 2281 W. 190TH STREET, SUITE 200, TORRANCE, CA, 90504, US

(21) Appl. No.: 12/863,585

(22) PCT Filed: Jan. 15, 2009
(86) PCT No.: PCT/AU2009/000038

§ 371 Date: Oct. 1, 2010

(30) Foreign Application Priority Data
Jan. 18, 2008 (AU) 2008900234
May 9, 2008 (AU) 2008902260

(51) Int. Cl. A61F 2/38

(52) U.S. Cl. 623/20.35

(57) ABSTRACT

An augment for use in a knee replacement system is described, the knee replacement system having a range of femoral components of different sizes, each different size of femoral component having a bone receiving region which is of predetermined dimensions. The augment has a proximal face and a distal face; the distal face is shaped to fit into and conform with the bone receiving region of a first femoral component; the proximal face is shaped to correspond with at least some of the predetermined dimensions of a bone receiving region of a second femoral component being of a smaller size than the first femoral component.

### TECHNICAL FIELD

The present invention relates to an augment for use in a knee replacement system and to a method of performing a knee replacement operation. The invention has particular application to revision knee replacement procedures.

### BACKGROUND TO THE INVENTION

Following a knee replacement operation, it sometimes becomes necessary to perform one or more revision knee replacement operations. This may be necessary because of loosening of one or both major components of the replacement knee. The components include the femoral component, which is placed on the end of the femur, and the tibial component, which is placed on the top of the resected proximal tibia. These two components are both formed from metal and may be affixed to bone ends with a layer of bone cement. Between these sits a polyethylene bearing. The height of the bearing varies according to the balance of the ligaments and the stability of the reconstruction.

The components may loosen due to wear of the plastic causing a condition known as osteolysis. The wear of the plastic creates microscopic particles, which then causes a cascade of events resulting in the bone between the cement and the bone, or between the prosthesis and the bone becoming extremely soft and allowing the prosthesis to undergo microscopic movement. This causes pain to the patient and a revision operation is required.

Revision may also be necessary following trauma, or following infection in the knee joint.

In a revision operation, the knee is opened and the components from the primary operation are removed. In doing this, in either a cement-free or cemented component, a certain amount of bone is removed. This may be soft bone due to osteolysis or soft bone due to stress shielding with the components not allowing the normal stress into the bone thereby causing the bone to become increasingly soft.

The new femoral component is selected from a range of standard sizes of femoral components. The femoral component used in a revision operation typically includes a stem for stability. This stem fits into the medullary canal.

When the revision procedure is performed, it is current practice to often use one or more augments on the new femoral component. The augments are small and generally rectangular and are formed from metal. The augments serve to space the femoral component away from the bone end to bring it down to a normal joint line. If these augments are not used then the level of the joint line may be unacceptably raised up more proximally. Typically, two distal femoral augments are used and sometimes one or more augments for the posterior femoral condyles are used in addition. The bone end is then resected to match the positioning of the augments. In practice, placing these augments and resecting the bone to match the positions of the augments becomes a complicated and time consuming procedure. Further, large gaps are often created between the femoral component and the bone end, this can occur particularly in the anterior chamfer area. These gaps are filled with bone cement, but may detract from stability of the femoral component and thus a further revision knee replacement operation may soon become necessary.

### SUMMARY OF THE INVENTION

In a first aspect the present invention provides an augment for use in a knee replacement system, the knee replacement system having a range of femoral components of different sizes, each different size of femoral component having a bone receiving region which is of predetermined dimensions: the augment has a proximal face and a distal face; the distal face is shaped to fit into and conform with the bone receiving region of a first femoral component; the proximal face is shaped to correspond with at least some of the predetermined dimensions of a bone receiving region of a second femoral component being of a smaller size than the first femoral component.

The augment may be arranged so that in use it extends around a bone end from the posterior face of the bone to the anterior face.

The augment may include a posterior section, a distal resection, an anterior chamfer section and an anterior section.

The distal face may include projections which are arranged to fit into indentations in the bone receiving region of the first femoral component.

The proximal face may include a region of porous-ingrowth surface.

The augment may include hydroxyapatite.

The augment may be formed at least partially from titanium.

The second femoral component may be found in another range of femoral components which are of a different style to the first femoral component.

In a second aspect the present invention provides a method of performing a knee replacement operation including the steps of: selecting a first femoral component from a range of femoral components of different sizes, each different size of femoral component having a bone receiving region which is of predetermined dimensions; shaping a bone end to correspond with the dimensions of the bone receiving region of a second femoral component, the second femoral component being of a smaller size than the first femoral component; fitting the first femoral component to the bone end with an augment interposed between the bone end and the femoral component, the augment having a proximal face and a distal face; the distal face is shaped to fit into and conform with the bone receiving region of the first femoral component; wherein the proximal face is shaped to correspond with the predetermined dimensions of a bone receiving region of the second femoral component.

The augment may be selected to extend around the bone end from the posterior face of the bone to the anterior face.

### BRIEF DESCRIPTION OF THE DRAWINGS

An embodiment of the present invention will now be described, by way of example only, with reference to the accompanying drawings, in which:

FIG. 1 shows a view of an augment according to an embodiment of the invention, the augment is shown in an inverted position;

FIG. 2 is a side view of the augment of FIG. 1;

FIG. 3 is a view of the augment of FIG. 1 from above;

FIG. 4 is a rear view of the augment of FIG. 1, and partially rotated;

FIG. 5 shows the augment of FIG. 1 in its usual orientation being inserted into a femoral component;

FIG. 6 shows the augment and femoral component of FIG. 5 with the augment fully inserted; and

FIG. 7 is a side view of the augment and femoral component of FIG. 6.

### DETAILED DESCRIPTION OF THE PREFERRED EMBODIMENT

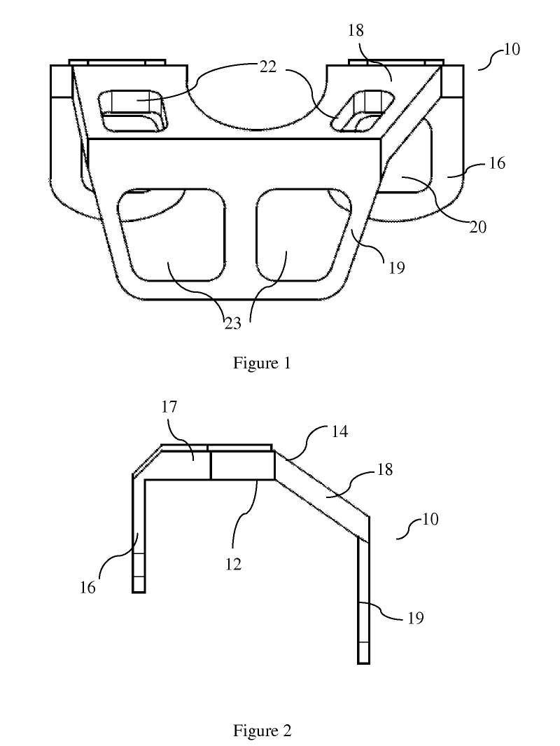

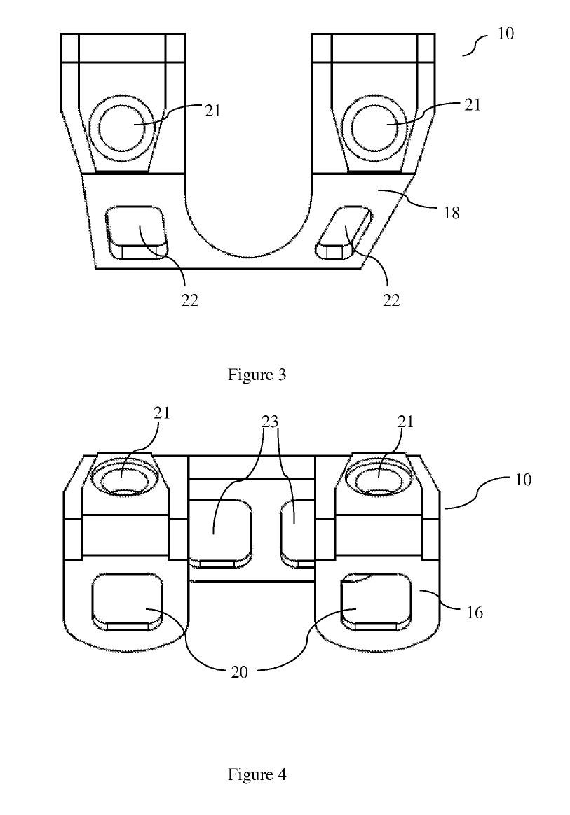

Referring to FIGS. 1 to 4, an augment 10 is shown for use in a knee replacement system. The augment 10 includes four generally straight sections being posterior section 16, distal resection 17, anterior chamfer section 18 and anterior section 19. Augment 10 has a proximal face 12 and a distal face 14. The proximal face 12 is the inner face of the augment and extends from the inside face of posterior section 16 to the inside face of anterior section 19. The distal face 14 is the outer face of the augment and extends from the outside face of posterior section 16 to the outside face of anterior section 19.

Distal resection 17 includes apertures 21 which receive screws to affix the augment 10 to a femoral component. Sections 16, 18 and 19 each include pairs of apertures, 20, 22 and 23 respectively. In use, these apertures can become filled with bone cement to achieve a solid bond between the femoral component and a bone end. The thickness of distal resection 17 is approximately 5 mm. The augment is formed from a suitable metal used for surgical prosthetics as are known in the art.

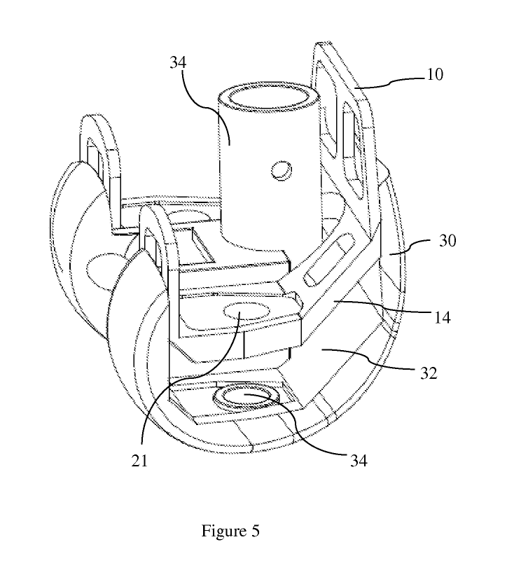

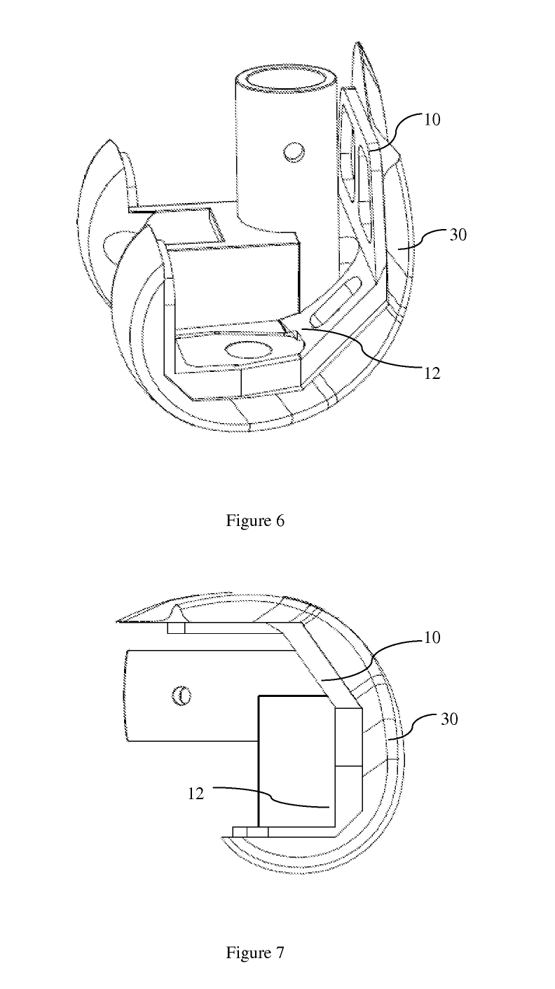

Referring to FIG. 5, a femoral component 30 is shown. Femoral component 30 is part of a knee replacement system, the system has a range of femoral components of different sizes, each different size of femoral component having a bone receiving region 32 which is of predetermined dimensions.

It can be seen that the distal face 14 of augment 10 is shaped to conform with the bone receiving region 32 of femoral component 30. The inner face of augment 10 is shaped to correspond with the bone receiving region of a smaller femoral component in the range of available femoral component sizes. In the illustrated example, the femoral component 32 is a size 4. However, the inner face of augment 12 corresponds to the dimensions of the bone receiving region of a size 3 femoral component, being one size smaller than a size 4. Augment 10 is secured in femoral component 30 by way of screws passing though apertures 21 of adaptor and being received in threaded holes 34 provided in component 30.

Use of the augment 10 will now be described with reference to the following example of a typical revision knee replacement operation.

Surgeon removes an existing primary size 4 femoral component from a patient. A size 4 revision femoral component 30 is selected as a suitable replacement. The surgeon prepares to resect the bone using secondary instruments which provide a template for resecting the bone end to particular predetermined sizes to correspond with the bone receiving regions of sizes of femoral components. The surgeon determines that the bone end has soft regions and that, if the bone end is resected to match a size 4, the bone end will not have sufficient strength and will also be too small to support size 4.

Traditionally, in this situation the surgeon would opt to use individual augments and cut out bone sections to match the augments as described above. However, the present invention provides the surgeon with an alternative. Instead of using individual augments, the surgeon elects to use an augment 10. The surgeon uses the standard secondary instruments to resect the bone end to a standard size 3. The surgeon then fits augment 10 to femoral component 30 by passing screws through apertures 21. The combination of augment 10 and component 30 are then fitted to the bone end with augment interposed between the bone end and the femoral component and with the bone end bearing against the proximal face 12 of augment 10. When fitted, the augment 10 extends around the bone end from the posterior face of the bone to the anterior face.

By using standard secondary resecting instruments to make all cuts, speed of preparing the bone end is increased. Further, the augment 10 is a good fit around the bone end, thus evenly distributing stress and providing good joint stability.

Augments according to the invention may be formed in various sizes. In the example described above, the thickness of the distal resection 17 was approximately 5 mm. Similarly, the augment can be formed in other thicknesses such as 10 mm or 15 mm. Also section 18 may be of different thicknesses.

In the example described above, the distal face of the adaptor conformed to a size 4 bone cavity, and the proximal face corresponded to a size 3 bone cavity. Similarly, the proximal and distal faces of the adaptor could correspond to other predetermined bone cavity sizes. For example, the distal face could correspond with size 6 and the proximal face could correspond with size 4. The augment effectively operates as an adaptor to change the dimensions of a bone receiving cavity of a femoral component from one size to another.

In the embodiment described above the augment extended in use from the anterior face of a bone end to the posterior face and was formed from four sections being the posterior section, the distal resection, the anterior chamfer section and the anterior section. In other embodiments, the augment may not extend so far around the bone end. For instance, the augment may have only three sections with either the anterior section or the posterior section being omitted. Still further, both of the posterior or anterior sections 16, 19 may be omitted. However, the embodiment with all four sections is the preferred form.

By using one-piece augments according to the invention, rather than numerous individual augments, the operation is made easier, faster and is less fiddly and a much more stable fit is achieved. The sacrifice of 1 or 2 millimetres of bone on the distal femur is overall of no great significance. The one-piece augment contacts the bone surface over a large area and spreads the load better than using individual augments. The anterior chamfer area becomes blocked by the augment leaving no large gaps as is the case with the prior art. Further, manufacturing costs are substantially lower.

In a cementable prosthesis, the surface of the knee prosthesis which is intended to contact the bone usually has small indentations in the surface which hold the bone cement. The augment may be manufactured to include projections which fit into these pockets and around the internal dimensions of the prosthesis and be screwed to the prosthesis. The area of the augment intended to contact bone may have a porous-ingrowth surface with or without hydroxyapatite to allow bone to adhere to the surface. In this way, the augment can be used to convert a cementable prosthesis to a cement free prosthesis. The augment may also incorporate a central-boss or stem with a porous surface to further increase prosthesis stability.

The apertures 20, 22 and 23 would be omitted in an augment that is intended to be fitted in a cement-free fashion, thus increasing the surface area for a porous-ingrowth surface. The apertures 21 would remain for affixing the augment to the component.

The augment may be formed from titanium, which has a degree of porosity and provides a porous-ingrowth surface. The surface may also be treated with hydroxyapatite to further encourage bone growth into the porous-ingrowth surface.

Augments according to the invention may similarly be used to convert a cement-free prosthesis to a cementable prosthesis.

The augment may be formed so that the outer surface of the augment is dimensioned to fit one of a range of prostheses, and the inner surface corresponds to the inner dimensions of a prosthesis of another style of prosthesis from another range produced by another manufacturer. For instance, the inner dimensions may correspond to a “Plus”™ prosthesis and the outer dimensions may correspond to a “Genesis”™ prosthesis. Thus, the bone end may be cut using the cutting jig for use with “Plus”™ prostheses and the augment used to allow fitting of a “Genesis”™ prosthesis. This allows the surgeon or practitioner greater flexibility in specifying prostheses and allows use of existing cutting tools to fit alternative types of prostheses.

Any reference to prior art contained herein is not to be taken as an admission that the information is common general knowledge, unless otherwise indicated.

Finally, it is to be appreciated that various alterations or additions may be made to the parts previously described without departing from the spirit or ambit of the present invention.

1. An augment for use in a knee replacement system, the knee replacement system having a range of femoral components of different sizes, each different size of femoral component having a bone receiving region which is of predetermined dimensions:
the augment has a proximal face and a distal face;
the distal face is shaped to fit into and conform with the bone receiving region of a first femoral component;
the proximal face is shaped to correspond with at least some of the predetermined dimensions of a bone receiving region of a second femoral component being of a smaller size than the first femoral component; wherein the augment is arranged so that in use it extends around a bone end from the posterior face of the bone to the anterior face.

2. An augment according to claim 1 which includes a posterior section, a distal resection, an anterior chamfer section and an anterior section.

3. An augment according to claim 1 wherein the distal face includes projections which are arranged to fit into indentations in the bone receiving region of the first femoral component.

4. An augment according to claim 1 wherein the proximal face includes a region of porous-ingrowth surface.

5. An augment according to claim 1 which includes hydroxyapatite.

6. An augment according to claim 1 which is formed at least partially from titanium.

7. An augment according to claim 1 wherein the second femoral component is found in another range of femoral components which are of a different style to the first femoral component.

8. An augment according to claim 1 wherein the proximal face is shaped to correspond with the predetermined dimensions of a bone receiving region of a second femoral component.

9. A method of performing a knee replacement operation including the steps of:
selecting a first femoral component from a range of femoral components of different sizes, each different size of femoral component having a bone receiving region which is of predetermined dimensions;
shaping a bone end to correspond with the dimensions of the bone receiving region of a second femoral component from the range, the second femoral component being of a smaller size than the first femoral component;
fitting the first femoral component to the bone end with an augment interposed between the bone end and the femoral component, the augment having a proximal face and a distal face;
the distal face is shaped to fit into and conform with the bone receiving region of the first femoral component;
wherein the proximal face is shaped to correspond with the predetermined dimensions of a bone receiving region of the second femoral component.

10. A method according to claim 9 wherein the augment is selected to extend around the bone end from the posterior face of the bone to the anterior face.
